(19) United States
(12) Patent Application Publication
(10) Pub. No.: US 2006/0253198 A1
(43) Pub. Date: Nov. 9, 2006

(54) Multi-lumen mold for intervertebral prosthesis and method of using same

(75) Inventors: Khin Myint, Shakopee, MN (US); Erik O. Martz, Savage, MN (US); Benjamin F. Carter, Eden Prairie, MN (US); Ronald Burke, Deephaven, MN (US)

Correspondence Address: FAEGRE & BENSON LLP;PATENT DOCKETING, 2200 WELLS FARGO CENTER, MINNEAPOLIS, MN, 55402, US

(73) Assignee: Disc Dynamics, Inc., Eden Prairie, MN (US)

(21) Appl. No.: 11/268,786

(22) Filed: Nov. 8, 2005

Related U.S. Application Data
(60) Provisional application No. 60/708,244, filed on Aug. 15, 2005.
(60) Provisional application No. 60/677,273, filed on May 3, 2005.
(60) Provisional application No. 60/708,245, filed on Aug. 15, 2005.

(51) Int. Cl. A61F 2/44

(52) U.S. Cl. 623/17.12; 623/17.16

(57) ABSTRACT

A multi-lumen mold is for the in situ formation of a prosthesis in an annulus located in an intervertebral disc space of a patient. The annulus has at least two openings formed by minimally invasive techniques and at least a portion of the nucleus pulposus is removed to form a nuclear cavity. The multi-lumen mold includes a mold adapted to be located in the nuclear cavity. A first lumen having a distal end is fluidly coupled to a flexible mold at a first location. At least a second lumen having a distal end fluidly coupled to the flexible mold at a second location. The first and second lumens are adapted to extend out through the openings in the annulus when the mold is positioned in the nuclear cavity. One or more securing members can be used to secure the mold in the intervertebral disc space. The securing members can engage with the annulus, the end plates, and/or another surface of a vertebrate.

The present application claims the benefit of U.S. Provisional Application Ser. No. 60/708,244 entitled Multi-Lumen Mold For Intervertebral Prosthesis And Method Of Using Same filed on Aug. 15, 2005; U.S. Provisional Application Ser. No. 60/677,273 entitled Catheter Holder for Spinal Implants filed May 3, 2005; and U.S. Provisional Application Ser. No. 60/708,245 entitled Catheter Holder for Spinal Implants filed Aug. 15, 2005, all of which are hereby incorporated by reference.

### FIELD OF THE INVENTION

The present invention relates to a multi-lumen approach to forming an intervertebral prosthesis in situ, and in particular to a multi-lumen mold for an intervertebral disc space adapted to receive an in situ curable biomaterial and a method of filling the mold.

### BACKGROUND OF THE INVENTION

The intervertebral discs, which are located between adjacent vertebrae in the spine, provide structural support for the spine as well as the distribution of forces exerted on the spinal column. An intervertebral disc consists of three major components: cartilage endplates, nucleus pulposus, and annulus fibrosus.

In a healthy disc, the central portion, the nucleus pulposus or nucleus, is relatively soft and gelatinous; being composed of about 70 to 90% water. The nucleus pulposus has a high proteoglycan content and contains a significant amount of Type II collagen and chondrocytes. Surrounding the nucleus is the annulus fibrosus, which has a more rigid consistency and contains an organized fibrous network of approximately 40% Type I collagen, 60% Type II collagen, and fibroblasts. The annular portion serves to provide peripheral mechanical support to the disc, afford torsional resistance, and contain the softer nucleus while resisting its hydrostatic pressure.

Intervertebral discs, however, are susceptible to disease, injury, and deterioration during the aging process. Disc herniation occurs when the nucleus begins to extrude through an opening in the annulus, often to the extent that the herniated material impinges on nerve roots in the spine or spinal cord. The posterior and posterolateral portions of the annulus are most susceptible to attenuation or herniation, and therefore, are more vulnerable to hydrostatic pressures exerted by vertical compressive forces on the intervertebral disc. Various injuries and deterioration of the intervertebral disc and annulus fibrosus are discussed by Osti et al., Annular Tears and Disc Degeneration in the Lumbar Spine, J. Bone and Joint Surgery, 74-B(5), (1982) pp. 678-682; Osti et al., Annulus Tears and Intervertebral Disc Degeneration, Spine, 15(8) (1990) pp. 762-767; Kamblin et al., Development of Degenerative Spondylosis of the Lumbar Spine after Partial Discectomy, Spine, 20(5) (1995) pp. 599-607.

Many treatments for intervertebral disc injury have involved the use of nuclear prostheses or disc spacers. A variety of prosthetic nuclear implants are known in the art. For example, U.S. Pat. No. 5,047,055 (Bao et al.) teaches a swellable hydrogel prosthetic nucleus. Other devices known in the art, such as intervertebral spacers, use wedges between vertebrae to reduce the pressure exerted on the disc by the spine. Intervertebral disc implants for spinal fusion are known in the art as well, such as disclosed in U.S. Pat. Nos. 5,425,772 (Brantigan) and U.S. Pat. No. 4,834,757 (Brantigan).

Further approaches are directed toward fusion of the adjacent vertebrate, e.g., using a cage in the manner provided by Sulzer. Sulzer's BAK® Interbody Fusion System involves the use of hollow, threaded cylinders that are implanted between two or more vertebrae. The implants are packed with bone graft to facilitate the growth of vertebral bone. Fusion is achieved when adjoining vertebrae grow together through and around the implants, resulting in stabilization.

Apparatuses and/or methods intended for use in disc repair have also been described for instance in French Patent Appl. No. FR 2 639 823 (Garcia) and U.S. Pat. No. 6,187,048 (Milner et al.). Both references differ in several significant respects from each other and from the apparatus and method described below.

Prosthetic implants formed of biomaterials that can be delivered and cured in situ, using minimally invasive techniques to form a prosthetic nucleus within an intervertebral disc have been described in U.S. Pat. No. 5,556,429 (Felt) and U.S. Pat. No. 5,888,220 (Felt et al.), and U.S. Patent Publication No. US 2003/0195628 (Felt et al.), the disclosures of which are incorporated herein by reference. The disclosed method includes, for instance, the steps of inserting a collapsed mold apparatus (which in a preferred embodiment is described as a “mold”) through an opening within the annulus, and filling the mold to the point that the mold material expands with a flowable biomaterial that is adapted to cure in situ and provide a permanent disc replacement. Related methods are disclosed in U.S. Pat. No. 6,224,630 (Bao et al.), entitled “Implantable Tissue Repair Device” and U.S. Pat. No. 6,079,868 (Rydell), entitled “Static Mixer”, the disclosures of which are incorporated herein by reference.

FIG. 1 illustrates an exemplary prior art catheter 11 with mold or balloon 13 located on the distal end. In the illustrated embodiment, biomaterial 23 is delivered to the mold 13 through the catheter 11. Secondary tube 11′ evacuates air from the mold 13 before, during and/or after the biomaterial 23 is delivered. The secondary tube 11′ can either be inside or outside the catheter 11.

### BRIEF SUMMARY OF THE INVENTION

The present invention relates to a method and apparatus for filling an intervertebral disc space with an in situ curable biomaterial using a multi-lumen mold. The present multi-lumen mold can be used, for example, to implant a prosthetic total disc, or a prosthetic disc nucleus, using minimally invasive techniques that leave the surrounding disc tissue substantially intact. The phrase intervertebral disc prosthesis is used generically to refer to both of these variations.

Minimally invasive refers to a surgical mechanism, such as microsurgical, percutaneous, or endoscopic or arthroscopic surgical mechanism, that can be accomplished with minimal disruption of the pertinent musculature, for instance, without the need for open access to the tissue injury site or through minimal incisions (e.g., incisions of less than about 4 cm and preferably less than about 2 cm). Such surgical mechanism are typically accomplished by the use of visualization such as fiber optic or microscopic visualization, and provide a post-operative recovery time that is substantially less than the recovery time that accompanies the corresponding open surgical approach.

Mold generally refers to the portion or portions of the present invention used to receive, constrain, shape and/or retain a flowable biomaterial in the course of delivering and curing the biomaterial in situ. A mold may include or rely upon natural tissues (such as the annular shell of an intervertebral disc) for at least a portion of its structure, conformation or function. The mold, in turn, is responsible, at least in part, for determining the position and final dimensions of the cured prosthetic implant. As such, its dimensions and other physical characteristics can be predetermined to provide an optimal combination of such properties as the ability to be delivered to a site using minimally invasive means, filled with biomaterial, prevent moisture contact, and optionally, then remain in place as or at the interface between cured biomaterial and natural tissue. In a particularly preferred embodiment the mold material can itself become integral to the body of the cured biomaterial.

The present mold will generally include both a cavity for the receipt of biomaterial and two or more conduits to that cavity. Some or all of the material used to form the mold will generally be retained in situ, in combination with the cured biomaterial, while some or all of the conduit will generally be removed upon completion of the procedure. Alternatively, the mold and/or lumens can be biodegradable or bioresorbable.

Biomaterial will generally refers to a material that is capable of being introduced to the site of a joint and cured to provide desired physical-chemical properties in vivo. In a preferred embodiment the term will refer to a material that is capable of being introduced to a site within the body using minimally invasive means, and cured or otherwise modified in order to cause it to be retained in a desired position and configuration. Generally such biomaterials are flowable in their uncured form, meaning they are of sufficient viscosity to allow their delivery through a cannula of on the order of about 1 mm to about 6 mm inner diameter, and preferably of about 2 mm to about 3 mm inner diameter. Such biomaterials are also curable, meaning that they can be cured or otherwise modified, in situ, at the tissue site, in order to undergo a phase or chemical change sufficient to retain a desired position and configuration.

The present method using the multi-lumen mold assembly of the present invention uses two or more discrete access points or annulotomies into the intervertebral disc space. The annulotomies facilitate performance of the nuclectomy, imaging or visualization of the procedure, delivery of the biomaterial to the mold through one or more lumens, drawing a vacuum on the mold before, during and/or after delivery of the biomaterial, and securing the prosthesis in the intervertebral disc space during and after delivery of the biomaterial.

The present multi-lumen mold is for the in situ formation of a prosthesis in an annulus located in an intervertebral disc space of a patient. The annulus has at least two openings formed by minimally invasive techniques and at least a portion of the nucleus pulposus is removed to form a nuclear cavity. The multi-lumen mold includes a mold adapted to be located in the nuclear cavity. A first lumen having a distal end is fluidly coupled to a flexible mold at a first location. At least a second lumen having a distal end fluidly coupled to the flexible mold at a second location. The first and second lumens are adapted to extend out through the openings in the annulus when the mold is positioned in the nuclear cavity. The first and second locations can optionally be located on generally opposite sides of the mold, on the same side of the mold, or a variety of other configurations.

One or more securing members can be used to secure the mold in the intervertebral disc space. The securing members can engage with the annulus, the end plates, and/or another surface of a vertebrate.

The present invention is also directed to a method for the in situ formation of a prosthesis in an annulus located in an intervertebral disc space of a patient. The method comprising the steps of forming at least first and second openings in the annulus using minimally invasive techniques. At least a portion of the nucleus pulposus is removed from the annulus to form a nuclear cavity. A mold is positioned in the annular cavity so that first and second lumens fluidly coupled to the mold extend through the first and second openings in the annulus. A biomaterial is delivered to the mold through one or more of the lumens. The biomaterial is permitted to at least partially cure. At least a portion of the first and second lumens extending out of the annulus are cut.

### BRIEF DESCRIPTION OF THE SEVERAL VIEWS OF THE DRAWING

FIG. 1 is an exemplary prior art catheter and mold.

FIG. 2 is a schematic illustration of various entry paths for use with the multi-lumen mold in accordance with the present invention.

FIG. 3A is a cross-sectional view of an annulus containing a multi-lumen mold in accordance with the present invention.

FIG. 3B is a cross-sectional view of the multi-lumen mold of FIG. 3A inflated with biomaterial.

FIG. 3C is a cross-sectional view of an intervertebral prosthesis formed from the multi-lumen mold of FIG. 3B.

FIG. 4 is a cross-sectional view of a multi-lumen mold with securing members in accordance with the present invention.

FIG. 5 is a cross-sectional view of a multi-lumen mold with alternate securing members in accordance with the present invention.

FIGS. 6A and 6B are cross-sectional views of a multi-lumen mold with alternate securing members in accordance with the present invention.

FIG. 6C is a cross-sectional view of the multi-lumen mold of FIG. 6C implanted posteriorly in accordance with the present invention.

FIG. 6D is a sectional view of an alternate embodiment of the multi-lumen mold assembly of FIG. 6A.

FIG. 6E is a side view of the multi-lumen mold of FIG. 6D.

FIG. 6F is a sectional view of an alternate embodiment of the multi-lumen mold assembly of FIG. 6A.

FIG. 7A is a cross-sectional view of a multi-lumen mold with securing members integrally formed with the mold in accordance with the present invention.

FIG. 7B is a side sectional view of the multi-lumen mold of FIG. 7A.

FIG. 8 is a cross-sectional view of a multi-lumen mold with a central tension member and securing members in accordance with the present invention.

FIGS. 9A and 9B are cross-sectional views of a multi-lumen mold with alternate securing members in accordance with the present invention.

FIG. 10A is a cross-sectional view of a multi-lumen mold with alternate securing features contained in a catheter in accordance with the present invention.

FIG. 10B is a cross-sectional view of the multi-lumen mold of FIG. 10A with the catheter partially withdrawn.

FIG. 10C is a cross-sectional view of the multi-lumen mold of FIG. 10B with the catheter fully withdrawn.

FIG. 11A is a cross-sectional view of a multi-lumen mold with pressure activated securing features in accordance with the present invention.

FIG. 11B is a cross-sectional view of a multi-lumen mold of FIG. 11A with the pressure activated securing features deployed in accordance with the present invention.

FIG. 12 is a cross-sectional view of an alternate multi-lumen mold used with a partial nuclectomy in accordance with the present invention.

FIG. 13 is a sectional view of a multi-lumen mold delivered to an intervertebral disc space through the adjacent vertebrae.

FIG. 14 is a sectional view of a multi-lumen mold delivered to an intervertebral disc space through an adjacent vertebrate and through the annulus.

FIG. 15 is a cross-sectional view of an alternate multi-lumen mold with securing members adapted to engage with the annulus and the adjacent vertebrae

FIG. 16 is a side sectional view of the alternate multi-lumen mold of FIG. 15.

FIG. 17 is a sectional view of a single lumen adapted for use with securing members in accordance with the present invention.

FIG. 18 is a sectional view of the single lumen mold of FIG. 17 combined with various securing members in accordance with the present invention.

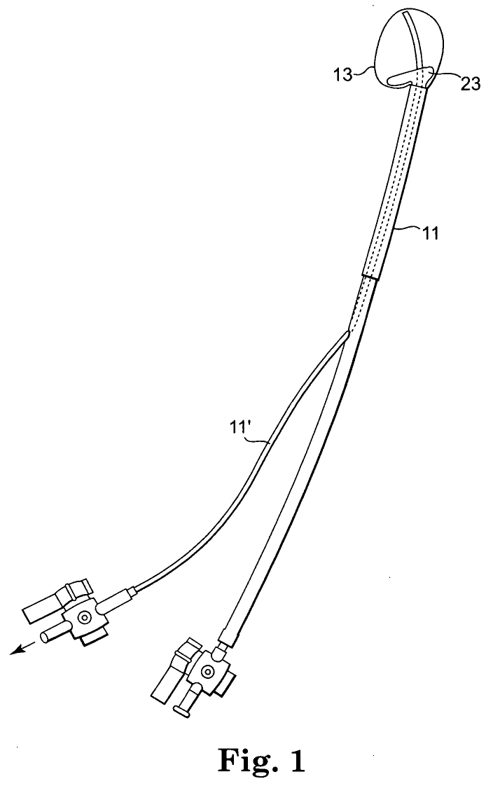

### DETAILED DESCRIPTION OF THE INVENTION

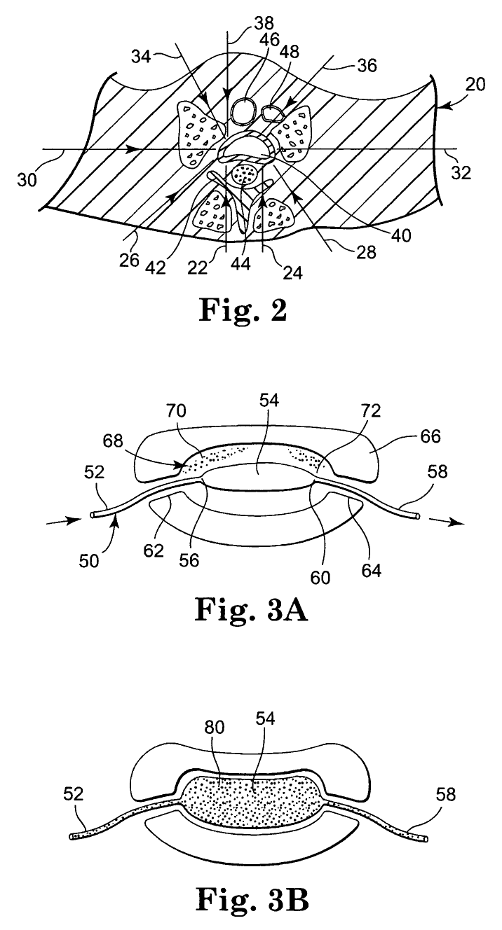

FIG. 2 is a cross-sectional view of a human body 20 showing various access paths 22 through 38 to the intervertebral disc 40 for performing the method of the present invention. The posterior paths 22, 24 extend either between or through the vertebrate 42 on either side of the spinal cord 44. The posterolateral paths 26, 28 are also on opposite sides of the spinal cord 44 but at an angle relative to the posterior paths 22, 24. The lateral paths 30, 32 extend through the side of the body. The anterior path 38 and anterolateral path 34 extend past the aorta 46, while the anterolateral path 36 is offset from the inferior vena cava 48.

The present method and apparatus use two or more of the access paths 22 through 38. While certain combinations of the access paths 22 through 38 may be preferred depending on a number of factors, such as the nature of the procedure, the patient's condition, etc., the present invention contemplates any combination of access paths.

In one embodiment, delivery catheter instruments are positioned along two or more of the access paths 22 through 38 to facilitate preparation of the intervertebral disc 40. Preparation includes, for example, formation of two or more annulotomies through the annular wall, removal of some or all of the nucleus pulposus to form a nuclear cavity, imaging of the annulus and/or the nuclear cavity, and positioning of the present multi-lumen mold in the nuclear cavity. In another embodiment, the present multi-lumen mold is positioned in the intervertebral disc 40 without use of delivery catheters.

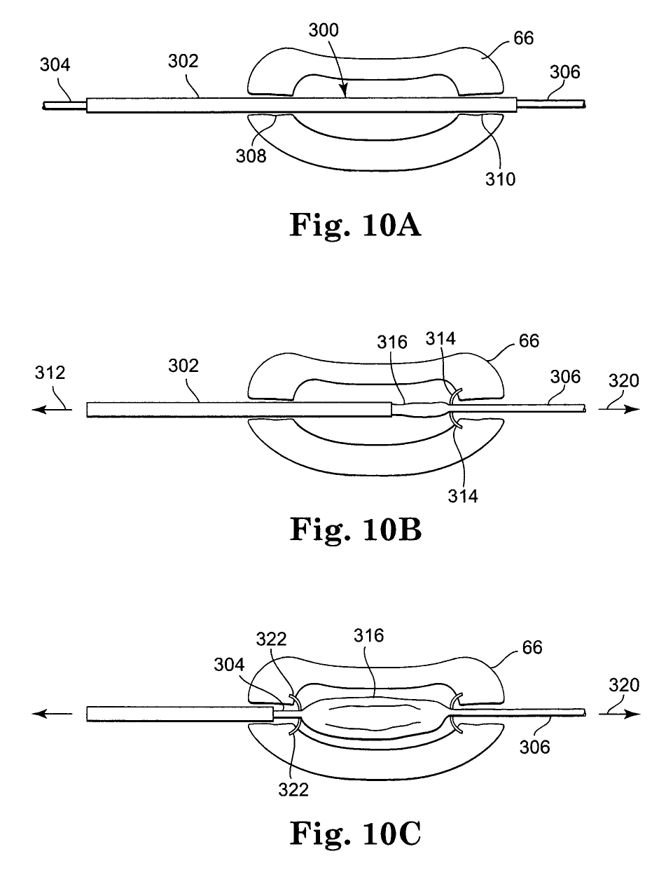

FIG. 3A illustrates a first embodiment of a multi-lumen mold assembly 50 in accordance with the present invention. The multi-lumen mold assembly 50 includes first lumen 52 fluidly coupled to mold 54 at location 56. Second lumen 58 is fluidly coupled to the mold 54 at location 60. Optional delivery catheters are not shown (see e.g., FIG. 10A). Various delivery catheters and catheter holders are disclosed in commonly assigned U.S. patent application Ser. No. ______, entitled Catheter Holder for Spinal Implants filed on the same date herewith (Attorney Docket No. 321296), which is hereby incorporated by reference.

The procedure involves forming annulotomy 62 at a first location in the annulus 66 and annulotomy 64 at a second discrete location. The nucleus pulposus 70 located in nucleus 68 is preferably substantially removed to create nuclear cavity 72. As illustrated in FIG. 3A, some portion of the nucleus pulposus 70 may remain in the nucleus 68 after the nuclectomy.

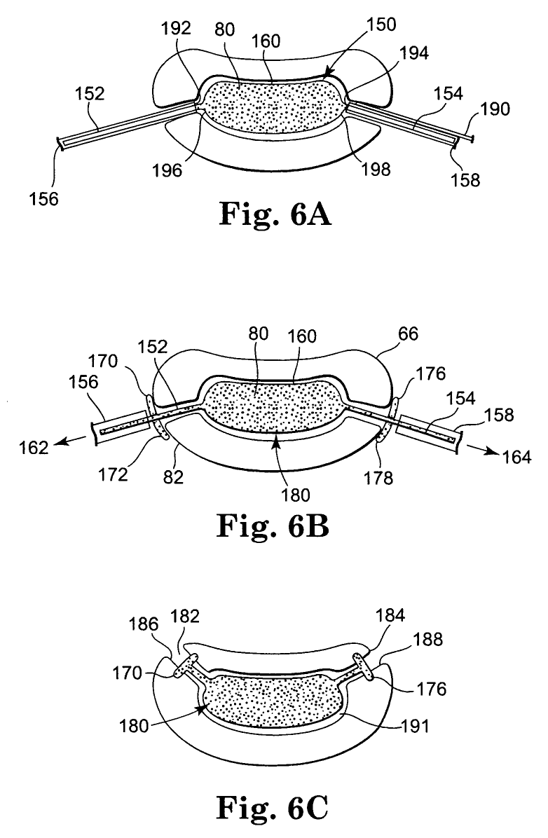

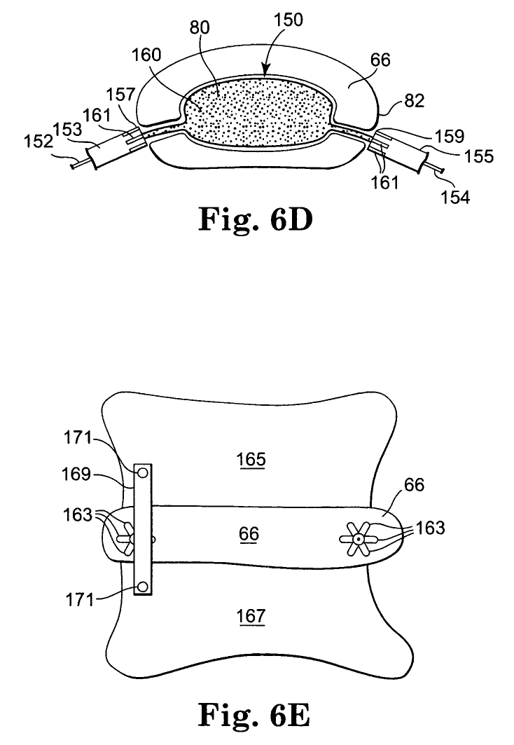

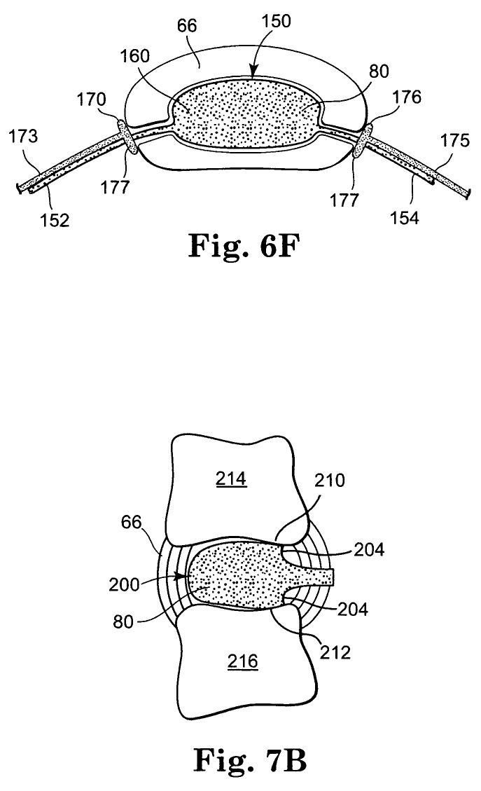

The multi-lumen mold assembly 50 is inserted through the annulotomies 62, 64 so that the mold 54 is positioned within the nuclear cavity 72. Alternatively, a catheter (see e.g., FIG. 6A) in one or both annulotomies 62, 64 can be used to insert the multi-lumen mold assembly 50 into the annulus 66. The mold 54 and/or lumens 52, 58 can optionally be biodegradable or bioresorbable.

For the sake of clarity, the annulotomies 62, 64 of FIG. 3A (and throughout this application) are schematically illustrated as having a cross-section greater than the diameters of the lumens 52, 58. The annulotomies 62, 64 can optionally have a cross-section less than, or equal to, the diameters of the lumens 52, 58. In practice, the tissue of the annulus 66 stretches to accommodate lumens 52, 58 with larger diameters than the cross section of the annulotomies 62, 64.

FIG. 3B illustrates the mold 54 substantially filled with biomaterial 80. The biomaterial 80 can be delivered to the mold 54 through the first lumen 52, the second lumen 58, or some combination thereof. In one embodiment, the biomaterial 80 is delivered through the first lumen 52 while a vacuum or reduced pressure condition is applied to the second lumen 58. In the illustrated embodiment, a portion of the biomaterial 80 is drawn into the second lumen 58 once the mold 54 is fully inflated.

As best illustrated in FIG. 3C, after the biomaterial 80 is at least partially cured, the first and second lumens 52, 58 are cut. In the illustrated embodiment, the lumen 52 is cut flush with the mold 54 at the location 56. The portions 86 of the second lumens 58 is cut flush with outer surface 82 of the annulus 66. While cutting the lumens 52, 58 flush with the mold 54 is preferred, a variety of method may be used. For example, in one embodiment the annulotomy 62 is an area of weakness in the annulus 66. The portion 58 can optionally be anchored to the annulus 66 to prevent the resulting prosthesis 88 from being expelled through the annulotomy 62.

FIG. 4 illustrates an alternate multi-lumen mold assembly 90 in accordance with the present invention. The first and second lumens 94, 98 are cut so that portions 96, 100 extend above the outer surface 82 of the annulus 66. Securing member 92A is attached to the portion 96 of the first lumen 94. Similarly, securing member 92B is attached to the portion 100 of the second lumen 98. Cured biomaterial 80 in the portions 96, 100 increases the tensile strength of the portions 96, 100. Alternatively, the lumens 94, 98 can be constructed from a rigid, semi-rigid, or pliable high tensile strength material.

Attachment of the securing members 92A, 92B can be achieved by a variety of techniques, such as adhesives, solvent bonding, mechanical deformation, mechanical interlock, or a variety of other techniques. In the illustrated embodiment, the securing members 92A, 92B include one or more prongs 102 which penetrate into the annulus 66 to further secure the resulting intervertebral prosthesis 104 within the nuclear cavity 72. In one embodiment, the securing members 92A, 92B include suture holes 93 that permit the surgeon to secure them to the annulus 66.

The resulting intervertebral prosthesis 104 is attached to the annulus 66 at two generally opposing locations. Consequently, the tendency for the intervertebral prosthesis 104 to be ejected through annulotomy 106 is resisted by the securing member 92B. Similarly, the tendency for the intervertebral prosthesis 104 to be ejected through annulotomy 108 is resisted by the securing member 92A.

The embodiment of FIG. 4 illustrates the securing members 92A, 92B located on the anterolateral portions of the annulus 66. This configuration restrains the prosthesis 91 from pressing against posterior wall 95 of the annulus 66. In one embodiment, the securing members 92A, 92B also engage with one or both of the adjacent vertebrae, such as illustrated in FIGS. 6E and 16.

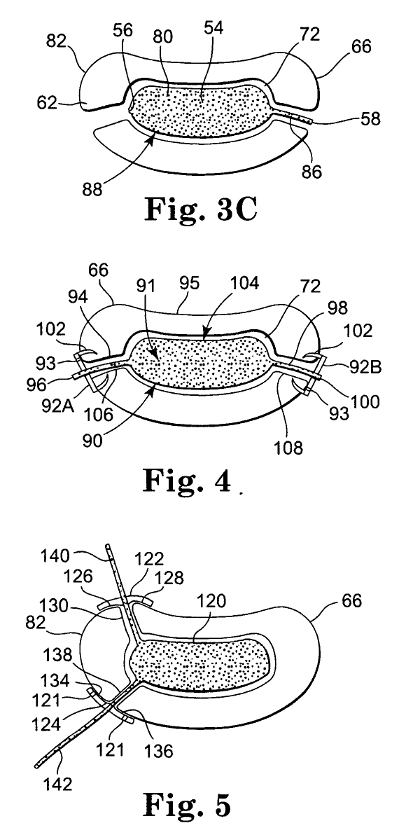

FIG. 5 illustrates an alternate multi-lumen mold assembly 120 with contoured securing members 122, 124 in accordance with the present invention. Securing member 122 includes a pair of curved surfaces 126, 128 that preferably match or approximate the outer surface 82 of the annulus 66 adjacent to the annulotomy 130. The securing member 122 is attached to the lumen 132 as discussed above. Similarly, the securing member 124 includes a pair of curved surfaces 134, 136 that matches or approximates the contour of the outer surface 82 of the annulus 66 adjacent to the annulotomy 138. After the securing members 122, 124 are secured to the multi-lumen mold 120, the distal ends 140, 142 are removed. The securing members 122, 124 optionally include suture holes 121, as discussed above. In one embodiment, the securing members 122, 124 are large enough to extend onto one or both of the adjacent vertebrae, and can optionally be secured thereto.

In the illustrated embodiment, the securing member 124 is located anterolateral and the securing member 122 is located posterolateral. The securing members 122, 124 are positioned to provide counteracting forces that resist displacement of the multi-lumen mold assembly 120.

FIGS. 6A and 6B illustrate an alternate multi-lumen mold assembly 150 in accordance with the present invention. The first and second lumens 152, 154 are preferably covered by catheters 156, 158, respectively. As discussed above, the biomaterial 80 can be delivered to the mold 160 through one or both of the lumens 152, 154. In one embodiment, the catheters 156 and/or 158 contain or are arranged co-linear with endoscopic visualization device 190 that permit the surgeon to assess the nuclectomy prior to delivery of the biomaterial 80. In another embodiment, distal ends 192, 194 of the catheters 156, 158, respectively, operate as tamps to shape portions 196, 198 of the mold 160.

As best illustrated in FIG. 6B, after delivery of the biomaterial 80 to the mold 160 is substantially completed, the catheters 156, 158 are retracted along a direction 162, 164, respectively. The exposed portion of the first lumen 152 preferably includes a securing member 170. Once the catheter 156 is retracted the biomaterial 80 flows into the securing member 170 to form protrusions 172 adjacent to the outer surface 82 of the annulus 66. Similarly, the lumen 154 includes a securing member 176 that also fills with biomaterial 80 when the catheter 158 is retracted. The securing member 176 is inflated with biomaterial 80 to form protrusions 178 adjacent to the outer surface 82 of the annulus 66.

In one embodiment, the securing members 170, 176 are part of the first and second lumens 152, 154. The securing members 170, 176 can be constructed from the same material as the mold 160 or a different material. In one embodiment, the securing members 170, 176 are spliced into the first and second lumens 152, 154 at a location adjacent to the outer surface 82 of the annulus 66. In another embodiment, the securing members 170, 176 are extensions of the mold 160.

When the biomaterial 80 is substantially cured, the catheters 156, 158 are removed and the lumens 152, 154 are cut at a location adjacent to the securing members 170, 176, securing the resulting intervertebral prosthesis 180 in the annulus 66.

FIG. 6C illustrates an alternate embodiment of the intervertebral prosthesis 180 of FIG. 6B, implanted through posterolateral annulotomies 182, 184. The securing members 170, 176 are positioned to engage with inside edges 186, 188 of the annulotomies 182, 184, respectively. Alternatively, the securing members 170, 176 can be positioned to engage with inside edges of the nuclear cavity 191 adjacent to the annulotomies 182, 184.

FIGS. 6D and 6E illustrate an alternate embodiment of the multi-lumen mold assembly 150 of FIG. 6A. The first and second lumens 152, 154 are preferably covered by catheters 153, 155, respectively. As discussed above, the biomaterial 80 is delivered to the mold 160 through one or both of the lumens 152, 154.

In the illustrated embodiment, distal ends 157, 159 of the catheters 153, 155 include a plurality of slits or openings 161 that selectively restrict inflation of the exposed portions of the lumens 152, 154 with the biomaterial 80. As the biomaterial 80 flows into the securing members 163, only the portions of the lumens 152, 154 adjacent to the slits 161 are permitted to inflate. As best illustrated in FIG. 6E, the slits 161 form a plurality of securing members 163 filed with biomaterial 80 in a pattern corresponding to the pattern of the slits 161. In the illustrated embodiment, the securing members 163 are formed adjacent to the outer surface 82 of the annulus 66. In an alternate embodiment, the securing members 163 can be formed to engage with the inner edges of the annulotomies, such as illustrated in FIG. 6C.

One or both of the securing members 163 can optionally be attached to adjacent vertebrae 165, 167 by securing member 169. In the illustrated embodiment, securing member 169 is a strap that is attached to the adjacent vertebrae 165, 167 using suitable fasteners 171. The strap 169 can be constructed from a rigid, semi-rigid or compliant biocompatible material.

FIG. 6F illustrate an alternate embodiment of the multi-lumen mold assembly 150 in accordance with the present invention. The first and second lumens 152, 154 are fluidly coupled to the mold 160. As discussed above, the biomaterial 80 can be delivered to the mold 160 through one or both of the lumens 152, 154. Separate lumens 173, 175 are fluidly coupled to securing members 170, 176. Consequently, the securing members 170, 176 can be inflated with fluid 177 independently of the delivery of the biomaterial 80 to the mold 160. Additionally, the fluid 177 can be another material, such as for example saline, air or a different biomaterial, than the biomaterial 80.

For some patients, it may be useful to inflate the securing members 170, 176 before delivery of the biomaterial 80 to the mold 160. After the biomaterial 80 is at least partially cured, the securing members 170, 176 can optionally be deflated and removed with the lumens 152, 154. In this embodiment, the securing members 170, 176 are temporary and only serve to secure the mold 160 in the annulus 66 during delivery of the biomaterial 80.

FIGS. 7A and 7B illustrate an alternate multi-lumen mold assembly 200 in accordance with the present invention. Mold 202 is configured to have one or more securing members 204 that are deployed when the mold 202 is inflated with biomaterial 80. In the illustrated embodiment, the securing members 204 comprise a curvilinear portions of the mold 202 positioned to engage with the inside surface 206 of the annulus 66. As best illustrated in FIG. 7B, the securing members 204 can also engage with the endplates 210, 212 of the adjacent vertebrae 214, 216.

FIG. 8 illustrates an alternate multi-lumen mold assembly 220 in accordance with the present invention. Mold 222 is formed around tension member 224. Center portion of the tension member 224 includes a plurality of openings 226 which fluidly communicate with the interior of the mold 222. First and second lumens 228, 230 are fluidly coupled to the tension member 224 and the inside of the mold 222.

As the biomaterial 80 is delivered through the lumens 228 and/or 230, it flows into the tension member 224 and through the openings 226 to inflate the mold 222. After the biomaterial 80 is at least partially cured, securing members 232, 234 are attached to distal ends 236, 238 of the tension member 224.

In the illustrated embodiment, the distal ends 236, 238 are designed to mechanically couple with the securing members 232, 234. The mechanical coupling can include threads, snap fit connections, or a variety of other mechanical structures. Once the securing members 232, 234 are secured to the tension member 224, the exposed portions of the first and second lumens 228, 230 are removed. Alternatively, the exposed portions of the first and second lumens 228, 230 can be removed before the securing members 232, 234 are secured to the tension member 224.

In an alternate embodiment, the securing members 232, 234 can be attached to the distal ends 236, 238 before the biomaterial 80 is delivered to the mold 222. This embodiment helps stabilize the position of the mold 222 relative to the annulus 66 during delivery of the biomaterial 80.

The tension member 224 is preferably flexible. The cured biomaterial 80 inside the tension member 224 is attached to the cured biomaterial in the mold 222 through the openings 226. The tension member 224 is anchored to the annulus 66 by the securing members 232, 234, resulting in a highly stable and secure intervertebral prosthesis 240. In one embodiment, the securing members 232, 234 are large enough to engage with the endplates of adjacent vertebrae, such as illustrated in FIG. 16.

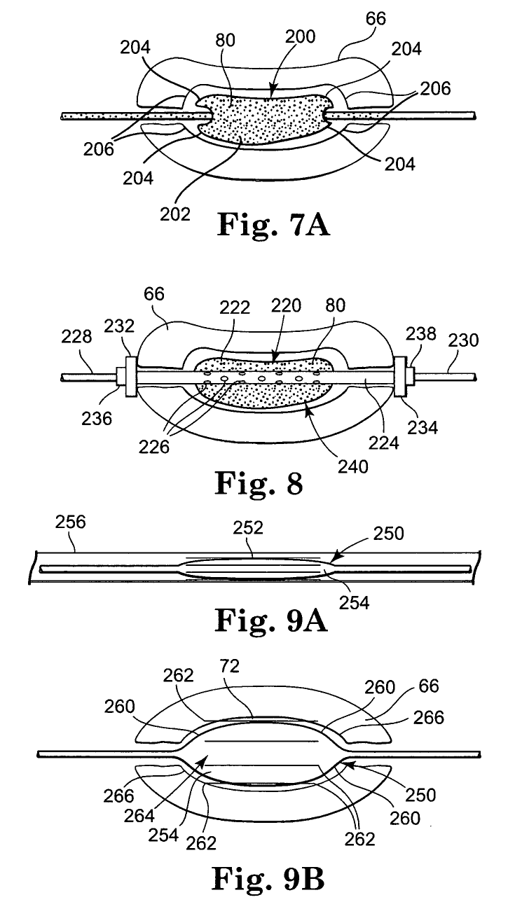

FIGS. 9A and 9B illustrate an alternate multi-lumen mold assembly 250 in accordance with the present invention. A plurality of rigid or semi-rigid securing members 252 are attached to the mold 254. The securing members 252 can be attached to the mold 254 using adhesives. In some embodiments the members are embedded into the mold 254. In one embodiment, the multi-lumen mold 250 is contained within a catheter 256 to facilitate insertion into an annulus 66.

FIG. 9B illustrates the multi-lumen mold 250 in an inflated state. The curvature 260 of the mold 254 causes distal ends 262 of the rigid members to project outwardly and engage with the inner surface of an annulus 66 to secure the resulting intervertebral prosthesis 264 in the nuclear cavity 72. In the preferred embodiment, the distal ends 262 also engage with the endplates of the adjacent vertebrae, such as illustrated in FIG. 16.

FIGS. 10A through 10C illustrate an alternate multi-lumen mold assembly 300 in accordance with the present invention. Catheter 302 extends around the first and second lumens 304, 306 to facilitate insertion of the multi-lumen mold assembly 300 through the annulotomies 308, 310 of the annulus 66. 5 As illustrated in FIG. 10B, the catheter 302 is partially retracted along the direction 312 to expose securing members 314 and a portion of the mold 316. Tension force 320 is preferably applied to the lumen 306 to drive the securing members 314 into the annular wall of the annulus 66 and/or the endplates of the adjacent vertebrae (see e.g., FIG. 16). 10 As illustrated in FIG. 10C, the catheter 302 is completely removed from the annulus 66 exposing securing members 322 while that tension force 320 is maintained. The annulus 66 preferably stretches or deforms in the direction of the tension force 320. When the tension force 320 is released, the natural resiliency of the annulus 66 drives the securing members 322 into the 1 5 opposite annular wall 66. The mold 316 is then inflated with biomaterial as discussed herein and the exposed portions of the lumens 304, 306 are removed.

In some embodiments, inflation of the mold 316 drives the securing members 314, 322 further into the annular wall 66. In an alternative embodiment, the securing members 314, 322 are driven into the annular wall 66 20 entirely by the forces generated during inflation of the mold 316. In another embodiment, the securing members 314, 322 engage with the endplates of adjacent vertebrae, such as illustrated in FIG. 16.

FIGS. 11A and 11B illustrate another embodiment of a multi-lumen mold assembly 350 in accordance with the present invention. Securing 25 members 352, 354 are located on opposite sides of the mold 356. The securing member 352 preferably includes a sleeve 358 that surrounds lumen 360. Flexible prongs 362 are integrally formed with the sleeve 358 and are oriented toward the mold 356. Similarly, securing member 354 includes a sleeve 364 that surrounds the lumen 366 with integrally formed flexible prongs 368.

In one embodiment, prongs 362, 368 are preferably sufficiently flexible to permit insertion of the multi-lumen mold 350 through the annulotomies 370, 372 without the use of a containing catheter. Alternatively, the multi-lumen mold 350 of FIG. 11A can be inserted into the annulus 66 while contained in a catheter, such as illustrated in FIG. 10A.

As illustrated in 11B, as the biomaterial 80 inflates the mold 356 the prongs 362, 368 are driven into the annular wall 66 and/or the endplates of the adjacent vertebrae (see e.g., FIG. 16). Once the biomaterial 80 is at least partially cured, the lumens 360, 366 are cut leaving the intervertebral prosthesis 380 securely positioned in the nuclear cavity 72. In another embodiment, the prongs 362, 368 engage with the endplates of adjacent vertebrae, such as illustrated in FIG. 16.

In another embodiment, reinforcing material 353 is located in the annular cavity 72, preferably along the posterior side of the annulus 66. The reinforcing material 353 can be a mesh, a film, a non-woven material made of metal, synthetics or combinations thereof. As the mold 356 inflates, the prongs 362, 368 engage the reinforcing material 353 to secure it between the securing members 352, 354 forming a sling that reinforces the posterior wall of the annulus 66. The reinforcing material 353 serves to transfer loads on the posterior wall of the annulus 66 to the securing members 352, 354.

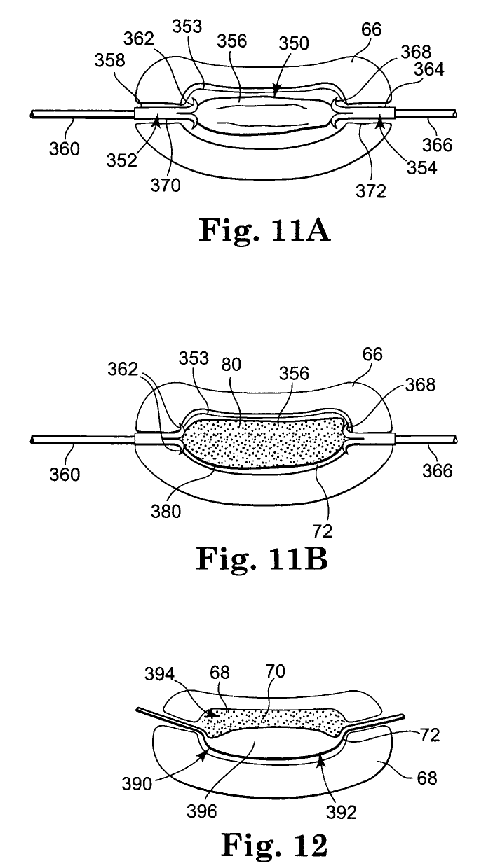

FIG. 12 illustrates an alternate multi-lumen mold assembly 390 used in connection with a partial nuclectomy in accordance with the present invention. In the embodiment of FIG. 12, the nucleus pulposus 70 located in nucleus 68 is only partially removed to create a nuclear cavity 72 in anterior region 392. Nucleus pulposus 70 remains in the posterior region 394 of the nucleus 68. Mold 396 is located in anterior region 392 of the nucleus 68. Any of the embodiments disclosed herein, including the various securing members, can be used with the partial nuclectomy method of FIG. 12.

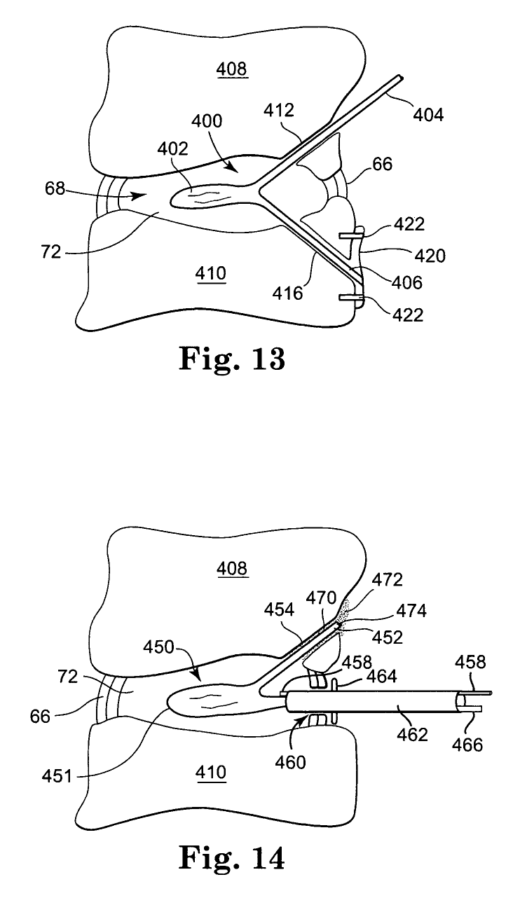

FIG. 13 illustrates an alternate multi-lumen mold assembly 400 in accordance with the present invention. The mold 402 includes first and second lumens 404, 406 configured to extend through the vertebrae 408, 410 and into the nucleus 68, without creating an annulotomy in the annulus 66. In particular, lumen 404 extends through a boring 412 that extends through vertebrate 408 and into nuclear cavity 72. Similarly, lumen 406 extends through boring 416 in the vertebrate 410 and into the nuclear cavity 72.

Once the mold 402 is filled with biomaterial, the lumens 404, 406 can be cut adjacent to the vertebrae 408, 410, adjacent to the nuclear cavity 72, or adjacent to the mold 402. In the embodiment of FIG. 13, securing member 420, such as a plate or a strap, is optionally attaches the lumen 406 and to vertebrate 410 using screws 422 or other suitable means. The securing member 420 can be constructed from a flexible or rigid biocompatible material, such as for example metals, plastics, ceramics, or composites thereof.

FIG. 14 illustrates an alternate multi-lumen mold 450 in accordance with the present invention. Lumen 452 extends through boring 454 to reach the nuclear cavity 72. Gap 470 is optionally filled with a securing material 472, such as for example an adhesive or bone cement to secure the lumen 452 to the vertebrate 408. In the illustrated embodiment, the securing material 472 is optionally spread over proximal end 474 of the lumen 452.

In the illustrated embodiment, the lumen 452 is cut flush with the vertebrate 408. In an alternate embodiment, the lumen 452 can be cut at the entrance to the nuclear cavity 72 or flush with mold 451.

Lumen 458 extends through annulotomy 460 in the annulus 66. Lumen 458 preferably extends through catheter 462, which includes a stop 464 that gauges the depth of penetration into the annulus 66. The catheter 462 optionally includes endoscope 466.

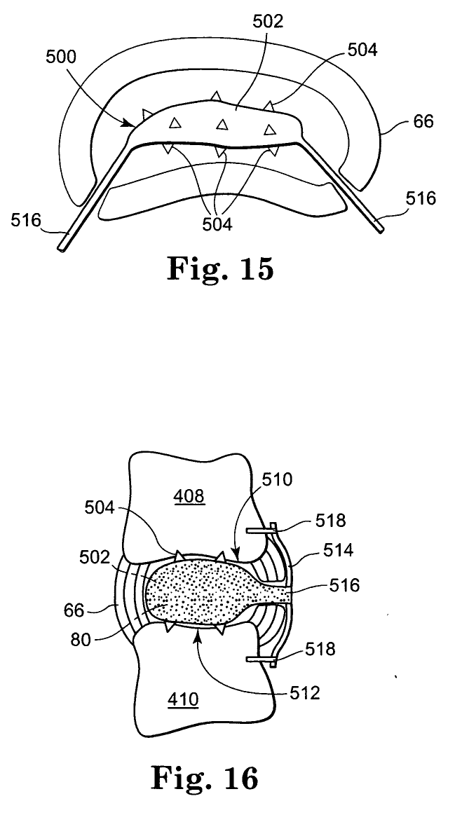

FIGS. 15 and 16 are an alternate multi-lumen mold assembly 500 in accordance with the present invention. The mold 502 includes a plurality of securing members 504. The securing members 504 can be integrally formed with the mold 502 or can attached thereto using a variety of techniques, such as for example adhesives, ultrasonic or solvent bonding, mechanical fasteners, and the like.

As best illustrated in FIG. 16, once the mold 502 is at least partially inflated with biomaterial 80, the securing members 504 engage with inner surface of the annulus 66 and/or the end plates 510, 512 of the adjacent vertebrae 408, 410, respectively. One or both of the lumens 516 are optionally attached to securing member 514, which is attached to the adjacent vertebrae 408, 410 using suitable fasteners 518.

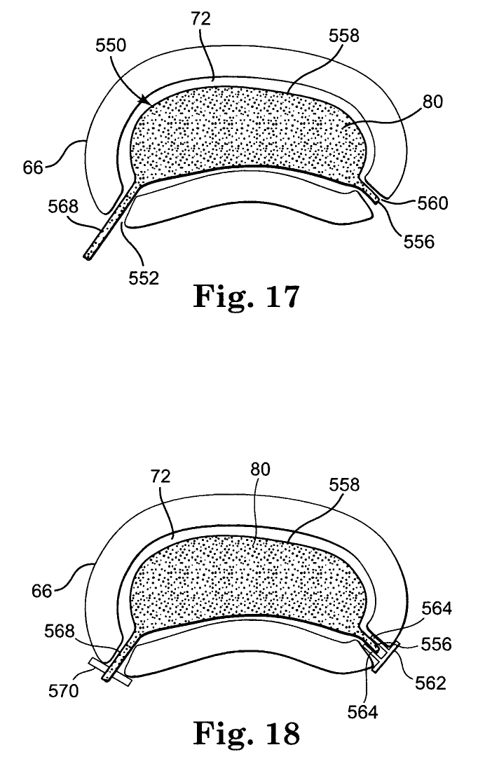

FIGS. 17 and 18 illustrate a single lumen mold assembly 550 used with exemplary securing mechanisms 562, 570 in accordance with the present invention. Any of the securing mechanisms disclosed herein can be used with the single lumen mold assembly 550 of FIGS. 17 and 18.

In the illustrated embodiment, portion 556 of mold 558 expands into second annulotomy 560 under pressure of the biomaterial 80. As best illustrated in FIG. 18, securing member 562 is attached to portion 556 of the mold 558. The securing member 562 can be attached using adhesives, mechanical fasteners, friction, and the like. In one embodiment, arms 564 compressively engage the portion 556 of the mold 558. The arms 564 optionally include barbed structures adapted to engage with the portion 556. Lumen 568 is cut and securing member 570 is attached. A variety of the securing members disclosed herein can be used with the lumen 568 and the portion 556.

In an alternate embodiment, portion 556 is preformed on the mold 558. The portion 556 is positioned in the annulotomy 560 before delivery of the biomaterial 80.

In yet another embodiment, the securing mechanism 562 is attached to the mold 558 before the mold 558 is positioned in the nuclear cavity 72. Lumen 568 is inserted into the annulotomy 560 and back out through annulotomy 552 until the securing member 562 is positioned against the annulus 66 as illustrated in FIG. 18. The mold 560 is then filled with biomaterial 80 and the lumen 568 is cut and/or secure as discussed herein.

The present multi-lumen molds can be used for performing the nuclectomy (removal of nucleus material); for evaluating the nuclectomy or the annulus 66; for imaging the annulus 66; for securing the mold during and after deliver of the biomaterial 80; and/or for delivering the biomaterial 80 to the mold. Disclosure related to evaluating the nuclectomy or the annulus and delivering the biomaterial 80 are found in U.S. patent application Ser. No. 10/984,493, entitled “Multi-Stage Biomaterial Injection System for Spinal Implants”, which is incorporated by reference. Various implant procedures and biomaterials related to intervertebral disc replacement suitable for use with the present multi-lumen mold are disclosed in U.S. Pat. No. 5,556,429 (Felt); U.S. Pat. No. 6,306,177 (Felt, et al.); U.S. Pat. No. 6,248,131 (Felt, et al.); U.S. Pat. No. 5,795,353 (Felt); U.S. Pat. No. 6,079,868 (Rydell); U.S. Pat. No. 6,443,988 (Felt, et al.); U.S. Pat. No. 6,140,452 (Felt, et al.); U.S. Pat. No. 5,888,220 (Felt, et al.); U.S. Pat. No. 6,224,630 (Bao, et al.), and U.S. patent application Ser. Nos. 10/365,868 and 10/365,842, all of which are hereby incorporated by reference.

The present multi-lumen mold can also be used with the method of implanting a prosthetic nucleus disclosed in a commonly assigned U.S. Patent Application entitled Lordosis Creating Nucleus Replacement Method And Apparatus (Attorney Docket No. 318946), filed on the same date herewith, the disclosure of which are incorporated herein by reference.

The multi-lumen mold and method of the present invention can also be used to repair other joints, including diarthroidal and amphiarthroidal joints. Examples of suitable diarthroidal joints include the ginglymus (a hinge joint, as in the interphalangeal joints and the joint between the humerus and the ulna); throchoides (a pivot joint, as in superior radio-ulnar articulation and atlanto-axial joint); condyloid (ovoid head with elliptical cavity, as in the wrist joint); reciprocal reception (saddle joint formed of convex and concave surfaces, as in the carpo-metacarpal joint of the thumb); enarthrosis (ball and socket joint, as in the hip and shoulder joints) and arthrodia (gliding joint, as in the carpal and tarsal articulations). The present multi-lumen mold can also be used for a variety of other procedures, including those listed above.

Patents and patent applications disclosed herein, including those cited in the Background of the Invention, are hereby incorporated by reference. Other embodiments of the invention are possible. Many of the features of the various embodiments can be combined with features from other embodiments. For example, any of the securing mechanisms disclosed herein can be combined with any of the multi-lumen molds. It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A multi-lumen mold for in situ formation of a prosthesis in an annulus located in an intervertebral disc space of a patient, the annulus having at least two openings formed by minimally invasive techniques and at least a portion of the nucleus pulposus removed to form a nuclear cavity, the multi-lumen mold comprising:
a mold adapted to be located in the nuclear cavity;
at least a first lumen having a distal end fluidly coupled to a flexible mold at a first location; and
at least a second lumen having a distal end fluidly coupled to the flexible mold at a second location, the first and second lumens are adapted to extend out through the openings in the annulus when the mold is positioned in the nuclear cavity.

2. The multi-lumen mold of claim 1 wherein the first and second locations are on generally opposite sides of the mold.

3. The multi-lumen mold of claim 1 wherein the first and second locations are on the same side of the mold.

4. The multi-lumen mold of claim 1 wherein the first lumen comprises a proximal portion adjacent to the annulus having at least one securing member adapted to secure the mold in the intervertebral disc space.

5. The multi-lumen mold of claim 4 wherein the securing member comprises engagement members that penetrate into the annulus adjacent to at least one of the openings.

6. The multi-lumen mold of claim 4 wherein the securing member comprises a shape that generally corresponds to a shape of an outer surface of the annulus.

7. The multi-lumen mold of claim 4 wherein the securing member comprises a flexible portion that inflates with biomaterial to engage with the annulus.

8. The multi-lumen mold of claim 7 comprising a catheter surrounding the securing member during delivery of the biomaterial to the mold.

9. The multi-lumen mold of claim 4 wherein the securing member is integrally formed with one of the first lumen or the mold.

10. The multi-lumen mold of claim 4 wherein the securing member comprises an extension of the mold.

11. The multi-lumen mold of claim 4 comprising at least one securing member securing the proximal end of the first lumen to one or more vertebrae adjacent to the intervertebral disc space.

12. The multi-lumen mold of claim 1 comprising at least one securing members attached to the mold proximate the first location to secure the mold in the intervertebral disc space.

13. The multi-lumen mold of claim 1 comprising:
a first securing member attached to the mold proximate the first location;
a second securing member attached to the mold proximate the second location; and
a reinforcing material attached to, and extending between, the first and second securing members within the nuclear cavity.

14. The multi-lumen mold of claim 1 wherein the mold comprises at least one securing member adapted to engage with one or more endplates on vertebrae adjacent to the intervertebral disc space or an interior surface of the nuclear cavity.

15. The multi-lumen mold of claim 14 wherein the securing members are one of attached to the mold or integrally formed in the mold.

16. The multi-lumen mold of claim 14 wherein the securing members are adapted for engagement when the mold is at least partially inflated with the biomaterial.

17. The multi-lumen mold of claim 1 comprising:
a tension member extending through an interior space of the mold, the tension member comprising distal ends extending through the openings in the annulus;
securing structures on the distal ends of the tension member adapted to receive at least one securing member; and
securing members adapted to engage with the securing structures on the distal ends of the tension member.

18. The multi-lumen mold of claim 1 comprising:
a plurality of securing members attached to one of the mold or the first and second lumens; and
a catheter retaining the securing members in a compressed state, such that removal of the catheter permits the securing members to resume an uncompressed state, the securing members positioned to engage with one or more endplates on one or more vertebrae adjacent to the intervertebral disc space or the annulus.

19. The multi-lumen mold of claim 18 wherein the securing members engage with one or more endplates on vertebrae adjacent to the intervertebral disc space or the annulus when the mold is at least partially inflated with biomaterial.

20. A method for the in situ formation of a prosthesis in an annulus located in an intervertebral disc space of a patient, the method comprising the steps of:
forming at least first and second openings in the annulus using minimally invasive techniques;
removing at least a portion of the nucleus pulposus from the annulus to form a nuclear cavity;
positioning a mold in the annular cavity so that first and second lumens fluidly coupled to the mold extend through the first and second openings in the annulus, respectively;
delivering a biomaterial to the mold through one or more of the lumens;
allowing the biomaterial to at least partially cure; and
cutting at least a portion of the first and second lumens extending out of the annulus.

21. The method of claim 20 comprising the step of locating the first and second lumens on generally opposite sides of the mold.

22. The method of claim 20 comprising the step of locating the first and second lumens on generally the same side of the mold.

23. The method of claim 20 comprising the step of securing a securing member to a proximal portion of the first lumen, the securing member having a cross sectional area greater than at least one of the openings in the annulus.

24. The method of claim 20 comprising engaging one or more securing members to secure the mold within the intervertebral disc space.

25. The method of claim 24 comprising penetrating the securing member into the annulus adjacent to at least one of the openings.

26. The method of claim 24 comprising engaging the securing member with an outer surface of the annulus.

27. The method of claim 24 comprising inflating the securing member with biomaterial to engage with the annulus.

28. The method of claim 27 comprising restraining inflation of the securing members with a catheter during delivery of the biomaterial to the mold.

29. The method of claim 24 comprising the step of integrally forming the securing member with the first lumen.

30. The method of claim 24 comprising the step of forming the securing member as an extension of the mold.

31. The method of claim 24 comprising the step of integrally forming the securing member with the mold.

32. The method of claim 20 comprising the steps of attaching a proximal portion on the first lumen to one or more vertebrae adjacent to the intervertebral disc space.

33. The method of claim 20 comprising engaging securing members on the mold to one or more endplates on vertebrae adjacent to the intervertebral disc space or an interior surface of the nuclear cavity.

34. The method of claim 33 comprising the step of at least partially inflating the mold with the biomaterial to engage the securing members with one or more endplates or an interior surface of the nuclear cavity.

35. The method of claim 20 comprising the steps of:
positioning a tension member through an interior space of the mold in the nuclear cavity; and
attaching securing members to distal ends of the tension member.

36. The method of claim 35 comprising the step of delivering biomaterial to the mold.

37. The method of claim 35 comprising the step of locating the securing members outside of the annulus.

38. The method of claim 20 comprising the steps of:
providing a plurality of securing members attached to one of the mold or the first and second lumens; and
retaining the securing members in a compressed state with a catheter;
removing the catheter to permit the securing members to resume an uncompress state; and
engaging the securing members with one or more endplates on vertebrae adjacent to the intervertebral disc space or the annulus.

39. The method of claim 38 comprising the step of at least partially inflating the mold with biomaterial to engage the securing members with the endplates or the annulus.

40. The method of claim 20 comprising the step of cutting portions of the first and second lumens flush with the mold.

41. The method of claim 20 comprising the step of cutting portions of the first and second lumens flush with an outer surface of the annulus.
